(19) United States
(12) Patent Application Publication
(10) Pub. No.: US 2024/0398949 A1
(43) Pub. Date: Dec. 5, 2024

(54) HER2 VARIANT CAR

(71) Applicant: OSLO UNIVERSITETSSYKEHUS HF, Oslo (NO)

(72) Inventors: Jon Amund KYTE, Hosle (NO); S. Esmaeil DORRAJI, Oslo (NO)

(21) Appl. No.: 18/698,327

(22) PCT Filed: Oct. 19, 2022
(86) PCT No.: PCT/EP2022/079110

§ 371 Date: Apr. 3, 2024

(30) Foreign Application Priority Data
Oct. 19, 2021 (GB) 2114938.0

(51) Int. Cl. A61K 39/00; C07K 14/725; C07K 16/22

(52) U.S. Cl. CPC A61K 39/464406; A61K 39/4611; A61K 39/4631; C07K 14/7051; C07K 16/22; C07K 2317/24; C07K 2317/33; C07K 2317/92

(57) ABSTRACT

The present invention provides a binding molecule which specifically binds p95HER2 comprising the amino acid sequence set forth in SEQ ID NO: 17, comprising a light chain variable domain (VL) and a heavy chain variable domain (VH) which together form an antigen binding unit, wherein the VL comprises three complementarity determining regions (CDRs): CDR1, CDR2 and CDR3, which respectively comprise the amino acid sequences SEQ ID NOs: 1, 2 and 3; and wherein the VH comprises three CDRs; CDR1, CDR2 and CDR3, which respectively comprise the amino acid sequences SEQ ID NOs: 4, 5 and 6.

### TECHNICAL FIELD OF THE INVENTION

The invention is related to the field of cancer therapy and diagnostics. In particular, it relates to novel targeting units and chimeric antigen receptors (CARs) comprising them, nucleic acids encoding the targeting units, nucleic acids encoding the CARS, immune cells expressing the CARs and their utility for treatment of cancer.

### BACKGROUND

Many HER2+ breast cancers express isoforms of HER2 with truncated carboxy-terminal fragments (CTF), collectively known as p95HER2.

Some antigen binding units for binding to p95HER2 are known, but not all of them are suitable for implementation into CARs. This is illustrated by the failures described in the Research Disclosure RD667070 published 17 Oct. 2019.

In order to achieve a therapeutic CAR-T cell (CAR-T), the cell needs to express the CAR in a sufficient amount in the cell membrane, and the antigen binding unit has to convey sufficient affinity and specificity for the target antigen. It can be expected that only a fraction of CAR-T cells with in vitro activity will successfully migrate to tumor metastases in vivo and/or infiltrate the hostile tumor microenvironment of a solid tumor. Furthermore, the CAR-T cells will likely need to sustain their activity over time in order to provide a therapeutic effect in vivo. It is therefore not trivial, but very desirable to obtain novel CARs able to provide a therapeutic effect for solid tumors in vivo when the CARs are expressed in the cell membrane of immune cells.

### SUMMARY

Provided herein are binding molecules comprising a novel antigen binding unit. Said antigen binding unit, and binding molecules comprising it, are able to specifically bind to cells expressing the hyperactive 611-CTF isoform of p95HER2 under physiological conditions. Because p95HER2 is a truncated transmembrane receptor, it is not trivial to obtain such antigen binding units. The antigen binding units herein display little or no binding to full length HER2 under physiological conditions and they display little or no cross-reactivity to healthy tissue. An antibody comprising the novel antigen binding unit displayed an affinity (KD) to p95HER2 of approximately 2 nM.

As demonstrated herein, the antigen binding units are suitable for implementation into CARs, and they retain sufficient affinity and specificity for p95HER2 in this format. The CARs are well expressed in T cells, and their functionality is confirmed by in vitro experiments and their therapeutic effect is demonstrated in an in vivo model.

In a first aspect, we provide a binding molecule which specifically binds p95HER2 comprising the amino acid sequence set forth in SEQ ID NO: 17, comprising a VL and a VH which together form an antigen binding unit,
wherein the VL comprises three complementarity determining regions (CDRs): CDR1, CDR2 and CDR3 which respectively comprise the amino acid sequences SEQ ID NOs: 1, 2 and 3; and
wherein the VH comprises three CDRs: CDR1, CDR2 and CDR3 which respectively comprise the amino acid sequences SEQ ID NOs: 4, 5 and 6.

Said binding molecule may comprise a VH comprising the amino acid sequence set forth in SEQ ID NO: 7 or a sequence with at least 90% identity thereto, and a VL comprising the amino acid sequence set forth in SEQ ID NO: 8 or a sequence with at least 90% identity thereto. Said antigen binding unit may be a scFv.

In a second aspect, we provide a Chimeric Antigen Receptor (CAR) comprising an antigen binding unit according to the first aspect. The CAR may comprise a human CD8α hinge.

The CAR may comprise, from N-terminal to C-terminal, a human CD8α hinge, a human CD8α transmembrane domain, a human 4-1BB costimulatory domain and a human CD3ζ signaling domain.

In a third aspect, we provide a nucleic acid encoding a binding molecule according to the first aspect or a CAR according to the second aspect.

In a fourth aspect, we provide a vector comprising the nucleic acid of the third aspect.

In a fifth aspect, we provide a cytotoxic immune cell expressing a CAR according to the second aspect in its cell membrane.

In a sixth aspect, we provide a pharmaceutical composition comprising a binding molecule according to the first aspect, a nucleic acid according to the third aspect, a vector according to the fourth aspect or a cytotoxic immune cell according to the fifth aspect.

In a seventh aspect, we provide a method of treatment of cancer in a human patient comprising the step of administering the cytotoxic immune cell of the fifth aspect or the pharmaceutical composition of the sixth aspect.

In an eighth aspect, we provide a method of treatment of cancer in a human patient comprising the steps:
(a) obtaining a sample comprising cancer cells from the patient;
(b) analysing whether the cancer cells express p95HER2 by contacting the cells ex vivo with a binding molecule according to the first aspect further comprising a moiety suitable for detection; and
(c) administering an approved chemotherapy to the patient if the cancer cells are p95HER2 positive.

In a ninth aspect, we provide a method of diagnosing cancer comprising the steps:
(a) obtaining a sample comprising cells from a human patient;
(b) analysing whether the cells express p95HER2 by contacting the cells ex vivo with a binding molecule according to the first aspect, wherein the binding molecule comprises a moiety suitable for detection; and
(c) diagnosing the patient with cancer if the cells express p95HER2.

In a tenth aspect, we provide a binding molecule according to the first aspect, a CAR according to the second aspect, a cytotoxic immune cell according to the fifth aspect or a pharmaceutical composition according to the sixth aspect for use in therapy.

In an eleventh aspect we provide a binding molecule according to the first aspect, a CAR according to the second aspect, a cytotoxic immune cell according to the fifth aspect or a pharmaceutical composition according to the sixth aspect for use in the treatment of cancer, wherein the cancer expresses p95HER2.

In a twelfth aspect we provide a method of diagnosing cancer in a subject, the method comprising:
(a) contacting a sample of cells from the subject with a binding molecule as defined in any one of claims 1 to 4, wherein the binding molecule further comprises a detection moiety;
(b) determining whether the cells express p95HER2; and
(c) if the cells express p95HER2, diagnosing the patient with cancer.

The method of the twelfth aspect is thus an ex vivo method performed on a sample.

### BRIEF DESCRIPTION OF THE DRAWINGS

FIG. 1: Reactivity and specificity of antibody against p95HER2

Reactivity of the antibody of interest was tested using flow cytometry against a panel of 15 HER2+/− cell lines. The antibody only bound to the p95HER2-T47D cell line and was not reactive to the cell lines expressing full-length HER2 (HER2+SK-BR-3, MDA-MB-468, and A549), and HER2-breast cancer cell lines T47D, MCF-7, and MDA-MB-231. The antibody also was not reactive to any of the other tested malignant cell lines.

FIG. 2: Antibody binding affinity

p95HER2 peptide was used as an analyte with serial dilutions from 0.6 to 2500 nM to determine the antibody's binding affinity. A control antibody against the HER2 cytoplasmic domain (as the reference) and the antibody of interest were covalently immobilized onto the surface of two different flow cells on a sensor chip. The association (ka) rate increased with increasing p95HER2 peptide concentration. Bimolecular interaction model 1:1 showed a low equilibrium dissociation constant (KD=2 nM) for the antibody of interest with a high maximal binding response (Rmax) at 137 RU. Two independent experiments were performed.

FIG. 3: epitope mapping

(a) The map of serial overlapping synthetic peptides (triplicate) based on the p95HER2 extracellular domain. Consecutive peptides share 11 aa. (b) Based on signal intensity, the antibody of interest was reactive just to peptide 1, the only peptide containing MPIW (SEQ ID NO: 33) which is highlighted (c) in the 3D structure of full-length HER2. (d) The map of C-terminally extended p95HER2 peptide which has been truncated from N-terminal (peptide 1-20, each peptide is one aa shorter than previous peptide) or two amino acids substitution with alanines (peptide 21-39). (e) The signal intensity from truncated peptides showed that PIW is crucial for antibody binding, in which W is the axis. On the other hand, substitutions revealed that KFPDEE (SEQ ID NO: 34) is also required for antibody binding. The serial overlapping blot contained triplicate from each peptide and truncation and substitution blots contained duplicate from each peptide.

FIG. 4 (A) visualizes the percentage of T cells transduced with the p95HER2 CAR construct compared to non-transduced T cells (NT). (B) demonstrates p95HER2-CAR-Ts in vitro cytotoxicity. p95HER2-CAR-Ts, control CD19-CAR-Ts and non-transduced T cells were co-cultured with T47D-p95HER2 and T47D (p95HER2-negative) cells. p95HER2-CAR-Ts induced apoptosis in more than 90% of T47D-p95HER2 target cells overnight, but did not have any effect on T47D cells. Also, no cytotoxicity was observed in respect of CD19-CAR-Ts or non-transduced T cells co-cultured with either T47D-p95HER2 or T47D cells. The CD19-CAR-Ts comprise a CD19-specific scFv cloned into the same CAR backbone as the p95HER2 CAR, with identical hinge, transmembrane and intracellular domains.

FIG. 5 visualizes p95HER2-CAR-T cytokine production. Both CD4+ and CD8+p95HER2-CAR-T compartments expressed a significantly higher level of TNFα and IFNγ when co-cultured with T47D cells expressing p95HER2 compared to p95HER2-negative T47D cells. Non-transduced T cells did not express any cytokines when co-cultured with either p95HER2-positive T47D or p95HER2-negative T47D.

FIG. 6 visualizes the anti-tumor effect of p95HER2-CAR-Ts in vivo. The tumor control of p95HER2-CAR-Ts and CD19-CAR-Ts on tumor growth in NSG mice bearing an orthopedic p95HER2 breast cancer model was evaluated by bioluminescence in vivo imaging. CAR-Ts were injected 2 times intravenously (2 and 5 weeks after tumor implantation, indicated by arrows), 5 million CAR-Ts at each time point. p95HER-CAR-Ts demonstrated significant tumor control only 2 weeks after the first injection and eradicated p95HER+ tumors completely 5 weeks after the first injection. CD19-CAR-Ts did not show any effect on p95HER+ tumor progression. There was no difference in tumor growth between CD19-CAR-T and tumor-only groups. Data are reported as ±SEM of a representative experiment of three independent replicates (n=10).

FIG. 7 visualizes the number of CAR-Ts in 1 μl of circulating blood. The number of p95HER2-CAR-Ts increased during the time course through activation by interacting with target cells expressing p95HER2 antigen. The p95HER2-CAR-Ts persisted in vivo more than 10 weeks after intravenous injection. The same trend was not observed for CD19-CAR-Ts through interaction with target cells expressing p95HER2 antigen in vivo. A female mouse on average has 2.5-3.75 ml circulating blood.

FIG. 8

Some breast cancer cells express isoforms of HER2 (sequence depicted-SEQ ID NO: 71) that are generated through two different mechanisms. Proteolytic cleavage of HER2 by metalloproteinases was the first mechanism to be discovered. The second mechanism involves the alternative initiation of translation from internal methionine codons located at positions 611, 648, 676 or 687. A number of isoforms with varying status of activity have been identified and are collectively referred to as p95HER2. The most potent and hyperactive p95HER2 isoform is called 611-HER2-CTF (carboxy-terminal fragment).

### DETAILED DESCRIPTION

Binding molecules comprising the novel antigen binding units herein generally comprise or consist of one or more proteins (i.e. polypeptide chains) and may have any suitable format including antibodies, scFv's, Fab's, immunotoxins, immunoconjugates, bispecific antibodies, CARs etc. Thus in an embodiment the binding molecule provided herein is an antibody or a fragment (that is, antigen-binding fragment) thereof. Examples of antigen binding fragments of antibodies include Fab, Fab′ and F (ab)′₂ moieties. In another embodiment the binding molecule is a scFv. In still another embodiment the binding molecule is a CAR.

The binding molecule provided herein specifically binds p95HER2 comprising the amino acid sequence PIWKFPDEE as set forth in SEQ ID NO: 17. As further detailed below, SEQ ID NO: 17 is the epitope recognised by the binding molecules provided herein.

Such binding molecules, especially soluble binding molecules such as antibodies, antigen-binding fragments of antibodies and scFvs, may be used in their “naked” form (i.e. not conjugated to a second agent) to target cancer cells. Alternatively such binding molecules may carry (e.g. be conjugated to) a toxic payload, e.g. a cytotoxin (such as saporin or gelonin) or a moiety comprising a radioactive isotope such as ¹⁷⁷Lu, ²²⁴Ra or ²²⁵Ac. A binding molecule conjugated to a toxic payload may be referred to as an immunotoxin.

Furthermore, the novel antigen binding units may be used as diagnostic agents, e.g. in the form of naked antibodies or binding molecules comprising a detectable label like a fluorescent or radioactive moiety. A detectable label may be referred to as a detection moiety, or a moiety suitable for detection.

We have measured a low equilibrium dissociation constant (KD=2 nM) for an antibody comprising the heavy chain variable region (VH) (SEQ ID NO: 7) and light chain variable region (VL) (SEQ ID NO: 8), with a high maximal binding response (Rmax) at 137 RU. The target epitope of the antigen binding units herein, is believed to be the sequence PIWKFPDEE (SEQ ID NO: 17). Said epitope is located in the p95HER2 isoform called 611-HER2-CTF (SEQ ID NO: 20).

(SEQ ID NO: 20)
MPIWKFPDEEGACQPCPINCTHSCVDLDDKGCPAEQRASPLTSIISAVV
GILLVVVLGVVFGILIKRRQQKIRKYTMRRLLQETELVEPLTPSGAMPN
QAQMRILKETELRKVKVLGSGAFGTVYKGIWIPDGENVKIPVAIKVLRE
NTSPKANKEILDEAYVMAGVGSPYVSRLLGICLTSTVQLVTQLMPYGCL
LDHVRENRGRLGSQDLLNWCMQIAKGMSYLEDVRLVHRDLAARNVLVKS
PNHVKITDFGLARLLDIDETEYHADGGKVPIKWMALESILRRRFTHQSD
VWSYGVTVWELMTFGAKPYDGIPAREIPDLLEKGERLPQPPICTIDVYM
IMVKCWMIDSECRPRFRELVSEFSRMARDPQRFVVIQNEDLGPASPLDS
TFYRSLLEDDDMGDLVDAEEYLVPQQGFFCPDPAPGAGGMVHHRHRSSS
TRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQP
PSPREGPLPAARPAGATLERPKTLSPGKNGVVKDVFAFGGAVENPEYLT
PQGGAAPQPHPPPAFSPAFDNLYYWDQDPPERGAPPSTFKGTPTAENPE
YLGLDVPV

Accordingly, we provide antigen binding units able to specifically bind to the sequence PIWKFPDEE (SEQ ID NO: 17) under physiological conditions. In an embodiment, a binding molecule, e.g. an antibody, comprising an antigen binding unit as provided herein has a KD of at least 2 nM.

As used herein, an “antigen binding unit” is a moiety comprising or consisting of one or more proteins, or parts thereof, able to bind an extracellular target epitope under physiological conditions. The antigen binding units herein may be able to bind an extracellular target epitope under physiological conditions in a tumor environment. The antigen binding units herein can specifically bind to p95HER2 expressed on cancer cells under physiological conditions. That is, the antigen binding units herein display little or no binding to full length HER2 under physiological conditions. Furthermore, the antigen binding units herein display little or no cross-reactivity to healthy tissue.

In particular, the antigen binding units herein can bind to epitopes that are masked in full-length HER2, but are exposed in 611-CTF. This makes them highly specific against the hyperactive p95HER2 isoform. Notably, 611-CTF is the only known isoform of p95HER that extensively induces expression of genes involved in metastasis and development of malignancy.

Binding molecules comprising the antigen binding units provided herein thus specifically bind p95HER2 comprising the amino acid sequence set forth in SEQ ID NO: 17, and can thus bind (or target) cancer cells which express p95HER2 isoforms which comprise the epitope of SEQ ID NO: 17 (such as p95HER2-611-CTF). In particular, the CARs provided herein, which comprise such an antigen binding unit, can target cytotoxic cells expressing the CARs against such cancer cells in order to destroy them.

The antigen binding unit provided herein comprises an antibody light chain variable domain (VL) and an antibody heavy chain variable domain (VH). Such variable domains are well-known for skilled persons. The antigen binding unit of an antibody, comprising a VL and a VH, is called a Fv. An antigen binding unit as defined herein may comprise a single polypeptide chain comprising both the VL and VH sequences (e.g. as in the case of an scFv), or alternatively the VL and VH may be provided on separate polypeptide chains (as in an Fv).

Each VL and VH herein comprises three complementarity determining regions (CDRs) flanked by framework sequences. The framework sequences may be human, humanized or murine sequences. The six CDRs comprise or consist of the following sequences:

VL CDR1 (SEQ ID NO: 1):
KSSQSLLSSGNQKNNLA
VL CDR2 (SEQ ID NO: 2):
YASTRQS
VL CDR3 (SEQ ID NO: 3):
LQHYSSPYT
VH CDR1 (SEQ ID NO: 4):
DYFMN
VH CDR2 (SEQ ID NO: 5):
QIRNKNYNYATYFAESLEG
VH CDR3 (SEQ ID NO: 6):
LRYDY

The CDR sequences as specified above were determined using the Kabat system.

A Framework1 sequence is N-terminal to the CDR1, a Framework2 sequence is located between CDR1 and CDR2, while a Framework3 sequence is located between CDR2 and CDR3.

Accordingly, both a VL and VH can be roughly visualized as follows, with the CDRs boxed and the N-terminus indicated as N—:

N-

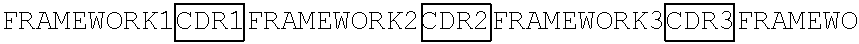

RK4

In one embodiment, the antigen binding unit comprises a murine VH comprising or consisting of the following sequence, in which the three CDRs are boxed (SEQ ID NO: 7):

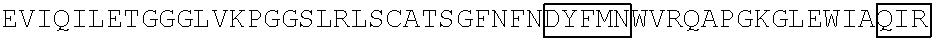

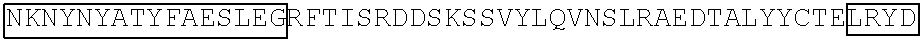

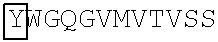

In another embodiment, the antigen binding unit comprises a VH comprising or consisting of a sequence with at least 90% or 95% identity to SEQ ID NO: 7.

In one embodiment, the antigen binding unit comprises a murine VL comprising or consisting of the following sequence, in which the three CDRs are boxed (SEQ ID NO: 8):

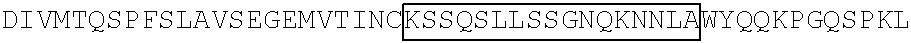

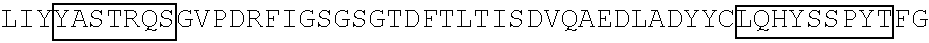

AGTKLELK

In another embodiment, the antigen binding unit comprises a VL comprising or consisting of a sequence with at least 90% or 95% identity to SEQ ID NO: 8.

The VH and VL may be connected by a disulphide bridge or a peptide linker. Alternatively, the two chains may be located within a Fab-fragment of an antibody (or any other antigen-binding fragment of an antibody) or an antibody as such. In one embodiment, the antigen binding unit comprises or consists of VL-linker-VH (from N- to C-terminus). In another embodiment, the antigen binding unit comprises or consists of VH-linker-VL (from N- to C-terminus). Such antigen binding units are often referred to as single chain Fv-fragments (scFv's). The linker has to have a certain length in order to allow the VH and VL to form a functional antigen binding unit. In one embodiment, the linker comprises 10 to 30 amino acid residues. In one embodiment, the linker comprises 15 to 25 amino acid residues, in particular glycine and/or serine residues.

In a particular embodiment the linker is a G₄S linker, i.e. a peptide linker comprising repeating units with the sequence GGGGS (SEQ ID NO: 21). For instance the linker may be a (G₄S)₃ (SEQ ID NO: 22), (G₄S)₄ (SEQ ID NO: 23) or (G₄S)₅ (SEQ ID NO: 24) linker (i.e. a linker comprising 3, 4 or 5 adjoining repeating G₄S units, respectively).

The linker may alternatively be a modified G₄S linker comprise one or more amino acid substitutions (optionally conservative amino acid substitutions, as defined below) in one or more G₄S units (preferably up to one amino acid substitution in one or more G₄S unit). In particular, a modified G₄S unit may comprise one or more substitutions of alanine for glycine. An example of a suitable linker as demonstrated below has the amino acid sequence set forth in SEQ ID NO: 18, which is a modified (G₄S)₄ linker in which one glycine residue has been substituted for alanine:

(SEQ ID NO: 18)
GGGGSGGGGSAGGGSGGGGS

In antigen binding units, the framework sequences may tolerate variation without destroying the specificity and affinity to the target antigen. For example, substitutions of amino acid residues may be tolerated better than deletions or additions of amino acid residues. Replacing murine framework sequences with human framework sequences, preferably of similar length, is known as humanization.

The term “conservative amino acid substitution”, as used herein, refers to an amino acid substitution in which one amino acid residue is replaced with another amino acid residue having a similar side chain.

Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus, a conservative amino acid substitution may be considered to be a substitution in which a particular amino acid residue is substituted for a different amino acid residue in the same family. In particular, the products comprising a conservative amino acid substitution relative to a reference sequence are covered by the terminology.

In one embodiment, each VL and VH herein comprises three CDRs flanked by human framework sequences. Human framework sequences are structurally conserved regions that normally tend to form a β-sheet structure delicately positioning the CDRs for specific binding to the target antigen under physiological conditions. Many human framework sequences are available from known human antibodies and from the international ImMunoGeneTics information system (IMGT) online database (see Giudicelli et al, Nucleic Acids Research, 2006, Vol. 34, Database issue D781-D784), but the term also covers human framework sequences comprising amino acid substitutions. Each of the human framework sequences may optionally comprise 0 to 5 amino acid substitutions relative to the natural sequence. An amino acid substitution is a sequence wherein an amino acid residue in a specific position is substituted for a different amino acid residue at the corresponding position, apparent when the sequences are aligned. Each of the human framework sequences may optionally comprise 1 amino acid substitution. Each of the human framework sequences may optionally comprise 2 or up to 2 amino acid substitutions. Each of the human framework sequences may optionally comprise 3 or up to 3 amino acid substitutions. Each of the human framework sequences may optionally comprise 4 or up to 4 amino acid substitutions. Each of the human framework sequences may optionally comprise 5 or up to 5 amino acid substitutions. The substitutions may be conservative substitutions. Even if such framework sequences are not necessarily previously known from human antibodies, they may provide lower immunogenic risk compared to a murine framework sequence. In one embodiment, 0 to 5 amino acid residues in the human framework sequences are substituted with the corresponding amino acid residue(s) from the murine parent sequences found in SEQ ID NOs: 7 and 8.

Collectively, scFv's comprising CDRs from a murine antibody and human framework sequences which each may optionally comprise 0 to 5 substitutions, are referred to as humanized scFv's. In some embodiments, some of the substitutions may be back to the parent murine amino acid residue (also known as “back mutations”).

In one embodiment, the human framework sequences are mature human framework sequences available from known human antibodies. Without being bound by theory, such framework sequences may convey very low risk of triggering unwanted immunogenic responses against the antigen binding unit, and at the same time increase the likelihood of obtaining stable binding units which are expressed well in cellular systems.

Generally speaking, in humanised VH and VL sequences the CDRs are not altered, and are retained, as in the parental VH and VL sequences. However as is known in the art, different programs, or schemes, are available to determine CDR sequences, and these may not in all cases give exactly co-incident results. Thus, different CDR identification schemes may yield different CDR sequences. For example, they may be shorter or longer, or positioned slightly differently in the VH or VL sequences (e.g. in a second scheme the CDR sequence may be partially displaced up- or downstream relative to a first scheme). Humanisation may be performed using a CDR grafting algorithm which uses different versions of the identified CDRs to transfer the CDRs from the original framework onto selected human sequences.

Thus, a humanised sequence may contain CDRs as identified according to any of the CDR identification schemes, e.g. the Kabat scheme, the IMGT scheme, and the Chothia scheme. The corresponding CDR sequences as determined by the IMGT and Chothia schemes are set out in Table 2 below. Thus, included herein are humanised VH and VL sequences, and antigen-binding units and binding molecules comprising them, which comprise CDR sequences as set out above or in Table 2 below. That is, a VH or VL sequence herein may comprise any of the sets of VH CDR1-3 or VL CDR1-3 as set out herein.

Examples of four different variants of the humanized VH are set forth in SEQ ID NOs: 72-75:

hu-VH1
(SEQ ID NO: 72)
EVQIVESGGGLVQPGGSLRLSCATSGFNFNDYFMNWVRQAPGKGLEWIA
QIRNKNYNYATYFAESVKGRFTISRDDSKSSVYLQMNSLKTEDTAVYYC
TELRYDYWGQGTMVTVSS
hu-VH2
(SEQ ID NO: 73)
EVQIVESGGGLVQPGGSLRLSCATSGFNFNDYFMNWVRQAPGKGLEWVA
QIRNKNYNYATYFAESVKGRFTISRDDSKNSVYLQMNSLKTEDTAVYYC
TELRYDYWGQGTMVTVSS
hu-VH3
(SEQ ID NO: 74)
QVQIQESGPGLVKPSETLSLTCTTSGFNFNDYFMNWVRQPPGKGLEWIA
QIRNKNYNYATYFAESLKSRFTISRDDSKSSVSLKLSSVTAADTAVYYC
TELRYDYWGQGTMVTVSS
hu-VH4
(SEQ ID NO: 75)
QVQIQESGPGLVKPSETLSLTCTTSGFNFNDYFMNWIRQPPGKGLEWIA
QIRNKNYNYATYFAESLKSRVTISRDDSKNQVSLKLSSVTAADTAVYYC
TELRYDYWGQGTMVTVSS

Examples of four different variants of the humanized VL are set forth in SEQ ID NOs: 76-79:

hu-VL1
(SEQ ID NO: 76)
DIVMTQSPDSLAVSLGERATINCKSSQSLLSSGNQKNNLAWYQQKPGQP
PKLLIYYASTRQSGVPDRFSGSGSGTDFTLTISSLQAEDVADYYCLQHY
SSPYTFGGGTKLEIK
hu-VL2
(SEQ ID NO: 77)
DIVMTQSPDSLAVSLGERATINCKSSQSLLSSGNQKNNLAWYQQKPGQP
PKLLIYYASTRQSGVPDRFSGSGSGTDFTLTISSLQAEDVAVYYCLQHY
SSPYTFGGGTKLEIK
hu-VL3
(SEQ ID NO: 78)
DIVMTQSPLSLPVTPGEPASISCRSSQSLLSSGNQKNNLAWYLQKPGQS
PQLLIYYASTRQSGVPDRFSGSGSGTDFTLKISRVEAEDVGDYYCLQHY
SSPYTFGGGTKLEIK
hu-VL4
(SEQ ID NO: 79)
DIVMTQSPLSLPVTPGEPASISCRSSQSLLSSGNQKNNLAWYLQKPGQS
PQLLIYYASTRQSGVPDRFSGSGSGTDFTLKISRVEAEDVGVYYCLQHY
SSPYTFGGGTKLEIK

In the foregoing humanized VH and VL sequences, the CDRs are as determined by the IMGT scheme as set out in Table 2 below.

In one embodiment, the antigen binding unit comprises the following combinations of humanized VH and VL sequences:

TABLE 1

| | |
|---|---|
| Humanized-v1 | hu-VH1 - huVL1 |
| Humanized-v2 | hu-VH1 - huVL2 |
| Humanized-v3 | hu-VH1 - huVL3 |
| Humanized-v4 | hu-VH1 - huVL4 |
| Humanized-v5 | hu-VH2 - huVL1 |
| Humanized-v6 | hu-VH2 - huVL2 |
| Humanized-v7 | hu-VH2 - huVL3 |
| Humanized-v8 | hu-VH2 - huVL4 |
| Humanized-v9 | hu-VH3 - huVL1 |
| Humanized-v10 | hu-VH3 - huVL2 |
| Humanized-v11 | hu-VH3 - huVL3 |
| Humanized-v12 | hu-VH3 - huVL4 |
| Humanized-v13 | hu-VH4 - huVL1 |
| Humanized-v14 | hu-VH4 - huVL2 |
| Humanized-v15 | hu-VH4 - huVL3 |
| Humanized-v16 | hu-VH4 - huVL4 |

More generally, in certain aspects, also provided herein is an antigen binding unit, or more particularly a binding molecule comprising an antigen-binding unit, which comprises the combinations of humanized VH and VL sequences as set out in Table 1.

In one embodiment, the antigen binding unit is or comprises an scFv comprising or consisting of the following sequence (SEQ ID NO: 9, CDRs boxed, linker in italics)

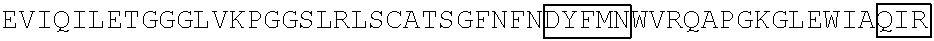

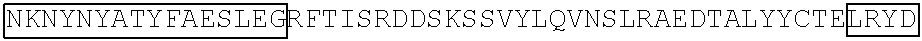

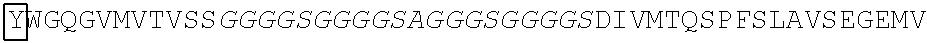

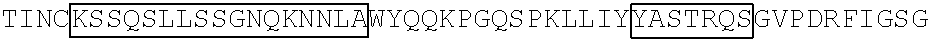

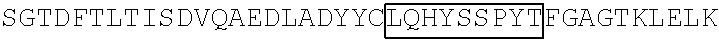

In another embodiment, the antigen binding unit is or comprises an scFv comprising or consisting of the amino acid sequence set forth in SEQ ID NO: 9, or an amino acid sequence with at least 90% or 95% sequence identity thereto.

In one embodiment, the antigen binding unit is or comprises an scFv comprising or consisting of the following sequence (SEQ ID NO: 10, CDRs boxed, linker in italics)

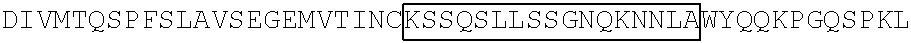

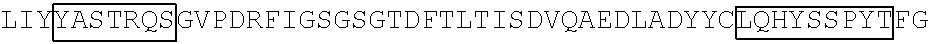

AGTKLELKGGGGSGGGGSAGGGSGGGGSEVQILETGGGLVKPGGSLRLSCAT

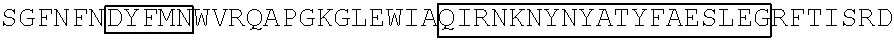

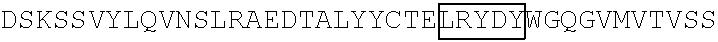

In another embodiment, the antigen binding unit is or comprises an scFv comprising or consisting of the amino acid sequence set forth in SEQ ID NO: 10, or an amino acid sequence with at least 90% or 95% sequence identity thereto.

As is apparent, SEQ ID NO: 9 comprises, from N-terminus to C-terminus, the VH of SEQ ID NO: 7, the linker of SEQ ID NO: 18 and the VL of SEQ ID NO: 8; SEQ ID NO: 10 comprises, from N-terminus to C-terminus, the VL of SEQ ID NO: 8, the linker of SEQ ID NO: 18 and the VH of SEQ ID NO: 7.

Novel chimeric antigen receptors (CARs) are provided. When the CARs herein are expressed on the surface of immune cells, such immune cells may be used in medicine. In particular, said immune cells may be used in treatment of solid tumors expressing p95HER2 comprising the amino acid sequence set forth in SEQ ID NO: 17. In one embodiment, said immune cells are used in treatment of p95HER2-positive breast cancer, p95HER2-positive gliomas or other p95HER2-positive cancers.

As used herein, CARs are artificial receptors comprising an extracellular antigen binding unit, a transmembrane domain and an intracellular signaling domain. The antigen binding unit in CARs is usually a scFv.

The antigen binding unit may be directly attached to the transmembrane domain. However, the CARs may comprise a hinge domain connecting the antigen binding unit to the transmembrane domain. The hinge domain may thus affect the steric conformation of the antigen binding unit. This may in turn affect the ability of the CAR to bind the target epitope and subsequently trigger signaling into an immune cell. If the target epitope is located too far from the cell membrane of the target cell or if the target epitope is otherwise hidden, the immune cell expressing the CAR may not be efficient. Accordingly, it is preferred that the target epitope is sufficiently accessible for immune cells expressing the CARs.

The transmembrane domain connects the extracellular domains to an intracellular signaling domain. Both the antigen binding unit and hinge domain are extracellular domains, i.e. they generally face the extracellular environment when expressed in the cell membrane of an immune cell. As used herein, “transmembrane domain”, means the part of the CAR which tends to be embedded in the cell membrane when expressed by an immune effector cell. Suitable transmembrane domains are well known for skilled persons. In particular, transmembrane domains from the human proteins CD8a, CD28 or ICOS may be used. The transmembrane domain is believed to convey a signal into immune cells upon binding of a target by the antigen binding unit.

The “intracellular signaling domain” refers to a part of the CAR located inside the immune cell when the CAR is expressed in the cell membrane. These domains participate in conveying the signal upon binding of the target. A variety of signaling domains are known, and they can be combined and tailored to fit the endogenous signaling machinery in the immune cells. In one embodiment the intracellular signaling domain comprises a “signal 1” domain like the signaling domains obtainable from the human proteins CD3ζ, FcR-γ, CD3ε etc. In general, it is believed that “signal 1” domains (e.g. the CD3ζ signaling domain) convey a signal upon antigen binding.

In another embodiment, the intracellular signaling domain further comprises a costimulatory domain. Such domains are well known and often referred to as “signal 2” domains, and they are believed to, subsequently to “signal 1” domains, convey a signal via costimulatory molecules. The “signal 2” is important for the maintenance of the signal and the survival of the cells. If absent, like in first-generation CARs, a CAR-T cell may be efficient in killing and in early cytokine release, but it will often become exhausted over time. Thus the intracellular signaling domain generally comprises both a “signal 1” and “signal 2” domain. Examples of such commonly used human “signal 2” domains include the 4-1BB signaling domain, CD28 signaling domain and ICOS signaling domain.

CARs in the present disclosure may comprise any of the antigen binding units as mentioned above. For example, CARs in the present disclosure may comprise an scFv comprising or consisting of the amino acid sequence set forth in SEQ ID NO: 9 or SEQ ID NO: 10, or a sequence with at least 90% or 95% identity thereto.

In particular, the CARs in the present disclosure may comprise any of the antigen binding units as mentioned above in the form of a scFv (e.g. the scFv of SEQ ID NO: 9 or SEQ ID NO: 10) connected to a CD8α hinge. The CD8α hinge is generally the human CD8α hinge of SEQ ID NO: 11, or a variant thereof with at least 90% or 95% sequence identity thereto.

SEQ ID NO: 11)
SDPTTTPAPRPPTPAPTIASQPLSLRPEACRPAAGGAVHTRGLDF

In particular, the CARs in the present disclosure may comprise an scFv as defined above (e.g. an scFv of SEQ ID NO: 9 or SEQ ID NO: 10) and an intracellular signaling domain comprising a CD3ζ signaling domain. The CD3ζ signaling domain is generally the human CD3ζ signaling domain of SEQ ID NO: 14, or a variant thereof with at least 90% or 95% sequence identity thereto.

(SEQ ID NO: 14)
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In a particular embodiment, in addition to the CD3ζ signaling domain the intracellular signaling domain further comprises a co-stimulatory domain, which may be any such domain as set out above, but in a particular embodiment is a 4-1BB co-stimulatory domain. The 4-1BB co-stimulatory domain is generally the human 4-1BB co-stimulatory domain of SEQ ID NO: 13, or a variant thereof with at least 90% or 95% sequence identity thereto.

(SEQ ID NO: 13)
KRGRKKLLYIFKQPFMRPVQTTQEEDGCSCRFPEEEEGGCEL

The CAR provided herein may in particular comprise a CD8α transmembrane domain, in particular the human CD8α transmembrane domain of SEQ ID NO: 12, or a variant thereof with at least 90% or 95% sequence identity thereto.

(SEQ ID NO: 12)
ACDIYIWAPLAGTCGVLLLSLVITLYC

In a particular embodiment the CAR comprises a CD8α hinge as described above and a CD8α transmembrane domain.

In particular, the CARs in the present disclosure may comprise the scFv of SEQ ID NO: 9 or SEQ ID NO: 10 connected to a CD8α hinge (SEQ ID NO: 11), wherein the CAR further comprises a CD8α transmembrane domain (SEQ ID NO: 12) and wherein the intracellular signaling domain comprises or consists of a 4-1BB costimulatory domain (SEQ ID NO: 13) and a CD3ζ signaling domain (SEQ ID NO: 14). Such a CAR has the amino acid sequence set forth in SEQ ID NO: 15:

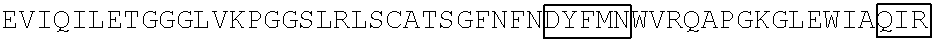

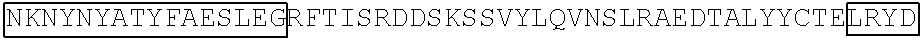

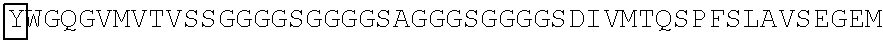

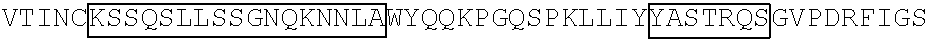

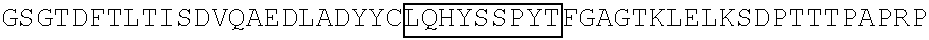

PTPAPTIASQPLSLRPEACRPAAGGAVHTRGLDFACDIYIWAPLAGTCGVLLLS
LVITLYCKRGRKKLLYIFKQPFMRPVQTTQEEDGCSCRFPEEEEGGCELRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPR

In a particular embodiment, the CAR provided herein comprises or consists of the amino acid sequence set forth in SEQ ID NO: 15, or a sequence with at least 90% or 95% identity thereto.

Sequence identity may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet., 16, (6) pp276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32 (5): 1792-1797, 2004) may be used for multiple sequence alignments, though any other appropriate programme may be used. Another suitable alignment programme is BLAST, using the blastp algorithm for protein alignments and the blastn algorithm for nucleic acid alignments. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively the standard EMBOSS Needle parameters may be used: matrix BLOSUM62, gap opening penalty 10, gap extension penalty 0.5. Any other suitable parameters may alternatively be used.

The immune cells expressing the CARs herein may be isolated from a patient or a compatible donor by leukapheresis or other suitable methods. Such primary cells may for example be T cells, NK cells or Macrophages. In particular, autologous T cells (both cytotoxic T cells, T helper cells or mixtures of these) may be transduced or transfected with nucleic acids encoding the CARs before a pharmaceutical composition comprising the cells is administered back to the patient. The immune cells expressing the CARs may also be cell lines suitable for clinical use like NK-92 cells. Generally, the immune cell expressing the CAR (whether a primary cell or a cell line) is a T cell (particularly a cytotoxic T cell) or an NK cell. Of course, the preferred cells are human when the intended patient is human.

The pharmaceutical composition herein can be a composition suitable for administration of therapeutic cells to a patient. The most common administration route for CAR T cells is intravenous administration. Accordingly, said pharmaceutical compositions may for example be sterile aqueous solutions with a neutral pH. For example, a patient's peripheral blood mononuclear cells may be obtained via a standard leukapheresis procedure. The mononuclear cells may be enriched for T cells, before transducing or transfecting them with a lentiviral vector or mRNA encoding the CARs. Said cells may then be activated with anti-CD3/CD28 antibody coated beads. The transduced/transfected T cells may be expanded in cell culture, washed, and formulated into a sterile suspension, which can be cryopreserved. If so, the product is thawed prior to administration.

In situations where the tumor is localized, different administrations methods may be used to improve efficacy. For example, regional or local administration rather than systemic administration of CAR-T cells might enhance efficacy.

The pharmaceutical compositions may comprise a pharmaceutically effective dose of the immune cells herein. A pharmaceutically effective dose may for example be in the range of 1×10⁶ to 1×10¹⁰ immune cells expressing the CARs. A pharmaceutically effective dose may for example be in the range of 1×10⁷ to 1×10⁹ T cells expressing the CARs. A pharmaceutically effective dose may for example be in the range of 1×10⁷ to 1×10⁹ NK cells expressing the CARs.

For efficient expression of the claimed CARs in immune cells, a conventional leader peptide may be introduced N-terminally for facilitating location in the cell membrane. One example of a suitable leader peptide is MESQTQALISLLLWVYGTYG (SEQ ID NO:16). The leader peptide is believed to be trimmed off and will likely not be present in the functional CAR in the cell membrane.

Accordingly, for expression of a second-generation CAR, nucleic acids encoding the following may be used: N-LEADER PEPTIDE-VH-LINKER-VL-HINGE-TRANSMEMBRANE DOMAIN-COSTIMULATORY DOMAIN-SIGNALING DOMAIN. Accordingly, for expression of a second-generation CAR, nucleic acids encoding the following may also be used: N-LEADER PEPTIDE-VL-LINKER-VH-HINGE-TRANSMEMBRANE DOMAIN-COSTIMULATORY DOMAIN-SIGNALING DOMAIN

The nucleic acids encoding the claimed CARs can be in the form of well-known RNA e.g. mRNA, or DNA expression vectors.

The pharmaceutical composition provided herein may alternatively be a composition suitable for administration of the binding molecule provided herein (e.g. antibody) to a patient. Such a composition generally comprises one or more pharmaceutically-acceptable excipients or suchlike, which are known in the art. A binding molecule as provided herein (such as an antibody), or a pharmaceutical composition comprising such a binding molecule, may be used in medicine/therapy, in particular to treat cancer expressing p95HER2 comprising the amino acid sequence set forth in SEQ ID NO: 17. The binding molecule or pharmaceutical composition may in particular be used to treat a solid cancer, e.g. breast cancer or glioma.

In one particular embodiment, there is provided a method of treatment of p95HER2 positive cancer in a human patient comprising the steps:
a. transducing or transfecting T cells, NK cells or Macrophages with mRNA encoding any of the CARs herein
b. repeatedly administering an effective dose of a pharmaceutical composition comprising said cells to a patient diagnosed with p95HER2 positive cancer.

In one particular embodiment, there is provided a method of treatment of p95HER2 positive cancer in a human patient comprising the steps:
a. transducing T cells, NK cells or Macrophages with mRNA encoding a p95HER2 CAR
b. repeatedly administering an effective dose of a pharmaceutical composition comprising said cells to a patient diagnosed with p95HER2 positive cancer.

In one particular embodiment, a method of treating a patient diagnosed with breast cancer is provided, wherein the method comprises the steps:
a. obtaining a sample comprising cancer cells from the patient;
b. analysing whether the cancer cells express p95HER2; and
c. administering a pharmaceutical composition comprising a pharmaceutically effective dose of T cells, NK cells or Macrophages expressing any of the CARs disclosed herein if the cancer cells are p95HER2 positive.

In one particular embodiment, a method of treating a patient diagnosed with breast cancer is provided, wherein the method comprises the steps:
a. obtaining a sample comprising cancer cells from the patient;
b. analysing whether the cancer cells express p95HER2; and
c. administering a pharmaceutical composition comprising a pharmaceutically effective dose of T cells, NK cells or Macrophages expressing a p95HER2 CAR if the cancer cells are p95HER2 positive.

In one particular embodiment, a method of treating a patient diagnosed with breast cancer is provided, wherein the method comprises the steps:
a. obtaining a sample comprising cancer cells from the patient;
b. analysing whether the cancer cells express p95HER2 by contacting the cells ex vivo with an antibody comprising a VL and a VH as described herein; and
c. administering a chemotherapy to the patient if the cancer cells are p95HER2 positive.

As noted above, the chemotherapy may be an approved chemotherapy. The chemotherapy may specifically target p95HER2 or cells expressing it.

In one particular embodiment, a method of diagnosing cancer in a human patient is provided, wherein the method comprises the steps
a. obtaining a sample comprising cells from the patient;
b. analysing whether the cells express p95HER2 by contacting the cells ex vivo with a binding molecule as provided herein, wherein the binding molecule comprises a moiety suitable for detection; and
c. diagnosing the patient with cancer if the cells express p95HER2.

In all the preceding aspects and embodiments, unless specified otherwise, the CDRs are identified using the Kabat scheme.

In another aspect, the CDRs may be identified using the IMGT or Chothia schemes. In particular, the IMGT and Chothia CDR sequences (SEQ ID NOs: 80-91) comprised within the murine VH (SEQ ID NO: 7) and murine VL (SEQ ID NO: 8) are set out in the following table:

TABLE 2

| | | CDRs in SEQ ID NO: 7 (VH) | CDRs in SEQ ID NO: 8 (VL) |
|---|---|---|---|
| IMGT | CDR1 | GFNFNDYF (SEQ ID NO: 80) | QSLLSSGNQKNN (SEQ ID NO: 83) |
| | CDR2 | IRNKNYNYAT (SEQ ID NO: 81) | YAS |
| | CDR3 | TELRYDY (SEQ ID NO: 82) | LQHYSSPYT (SEQ ID NO: 84) |
| Chothia | CDR1 | GFNFNDY (SEQ ID NO: 85) | KSSQSLLSSGNQKNNLA (SEQ ID NO: 88) |
| | CDR2 | RNKNYNYA (SEQ ID NO: 86) | YASTRQS (SEQ ID NO: 89) |
| | CDR3 | LRYDY (SEQ ID NO: 87) | LQHYSSPYT (SEQ ID NO: 90) |

In all the preceding aspects and embodiments, the CDRs may be the IMGT or Chothia CDRs as stated in this aspect.

### EXAMPLES

### Example 1: Antibody Specificity and Affinity

### Methods

### Cell Culture

Cell lines were cultured in DMEM (Sigma-Aldrich). Culture media were supplemented with 100 U/ml Penicillin-Streptomycin (Sigma-Aldrich) and 10% heat-inactivated fetal bovine serum (FBS) (Sigma-Aldrich). Cell lines were incubated at 37° C. with 5% CO₂ and 100% humidity.

Generation of p95HER2 Antibodies

Development of p95HER2 antibodies was done by immunizing rats with cells expressing 611-CTF-HER2. The immunization and hybridoma generation was performed by Aldevron (Freiburg, Germany). In brief, 8 to 12 weeks old rats were injected intradermally with 10 μg of immunization vector DNA expressing 611-CTF-HER2, fixed to gold particles. The selected hybridoma candidates were sub-cloned by limited dilution. Collected monoclonal hybridoma supernatants were used for final screening with flow cytometry and/or ELISA.

### Flow Cytometry

Cells were washed, pelleted down and resuspended in 100 μl FACS buffer (containing phosphate-buffered saline (PBS), pH 7, 2% FBS, and 2 mM EDTA). The antibody of interest (10 μg/ml) was added and incubated for 30 min at 4° C. in the dark. Cells were washed twice, resuspended in 100 μL FACS buffer containing secondary antibody goat anti-rat-PE (0.26 μg/ml) and Fixable Viability Dye eFluor™ 780 and incubated for 30 minutes in the dark at 4° C. Cells were washed, resuspended in 200 μL FACS buffer, and analyzed on a LSR II flow cytometer (BD Biosciences, Franklin Lakes, NJ, USA). Data were analyzed with FlowJo software (v10.7.1, FlowJo LLC).

### Surface Plasmon Resonance (SPR)

SPR was performed as described previously in Gomes & Andreu, Journal of Immunological Methods 259:217-230, 2002. In brief, control anti-HER2 (5 μg/ml) antibody and the antibody of interest (5 μg/ml) were covalently immobilized onto the surface of two different flow cells on sensor chip CM5 (2104988, GE Healthcare) using Amine Coupling Kit (BR-1000-50, GE Healthcare) and HBS-EP+ buffer. The extracellular domain of p95HER2 peptide with a polyhistidine-tag in the C-terminus (SEQ ID NO: 19) was used as analyte with serial dilutions from 0.6 to 2500 nM. MPIWKFPDEEGACQPCPINCTHSCVDLDDKGCPAEQRASPLTHHHHHH (SEQ ID NO: 19)

Kinetics of molecular interaction were processed by global curve fitting to the 1:1 bimolecular interaction model. Biacore T200 (GE Healthcare) was used to perform the experiment and all procedures were conducted at 25° C.

### Results

The generation of p95HER2-specific Abs was performed by immunizing rats with cells transfected with 611-HER2-CTF. To this aim, HEK-293 cells were transfected with different 611-HER2-CTF constructs, and the surface expression of p95HER2 was measured using an anti-tag antibody, and an irrelevant anti-tag antibody as a control. The immunization of rats for the generation of mAb hybridomas was done using cells transfected with pB1-611-CTF-hum.ECD. The initial screening of polyclonal hybridoma culture supernatants (HCS) against p95HER2 was performed using the Intellicyt iQue flow cytometry platform. The top nine positive clones were selected based on mean fluorescence intensity (MFI) values. The HCS from these nine polyclonal hybridomas (pClones) were then tested by flow cytometry for binding to the cell lines p95HER2-T47D, SK-BR-3, T-47D, and SUP-T1. HCS from pClones 1, 2, 3 and 8 were found to bind to p95HER2-T47D but not T47D. Only HCS from pClones 2 and 8 showed any binding to SK-BR-3, a cell line that expresses full-length HER2. To confirm these results, immunofluorescence (IF) staining was performed on p95HER2-T47D, SK-BR-3, and T-47D. Here, it was found that HCS from pClones 1, 2, and 3 stained p95HER2-T47D, but not T-47D or SK-BR3.

Based on these data, we chose pClones 1, 2, and 3 for subcloning into monoclonal cultures (mClones) through limited dilution series. The Intellicyt iQue screening of mClones 1, 2, and 3 demonstrated that only mClone 1 bound specifically to transfected cells, while mClone 2 bound non-specifically to non-transfected cells and mClone 3 was negative. To further test all three mClones, we performed flow cytometry analysis on the cell lines p95HER2-T47D, SK-BR-3 and T-47D using the HCS. The flow cytometry results confirmed the iQue screening data. Only mClone 1 bound specifically to p95HER2-T47D, while mClone 2 bound to both p95HER2-T47D and SK-BR-3. Furthermore, mClone 1 demonstrated stronger reactivity to p95HER2 compared to pClone 1 when assessed by IF. Based on these screening results, mClone 1 was selected for generating a mAb (termed the Oslo-2 antibody herein).

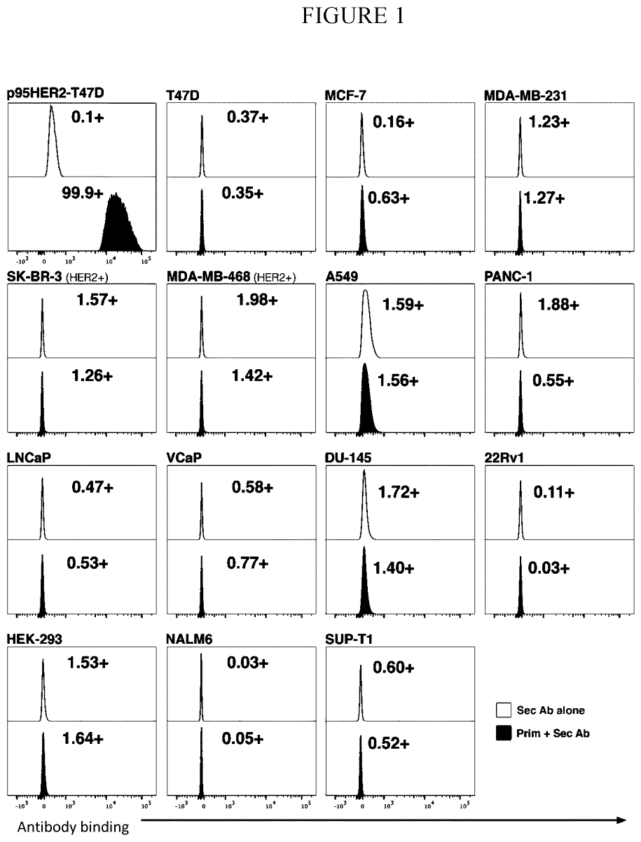

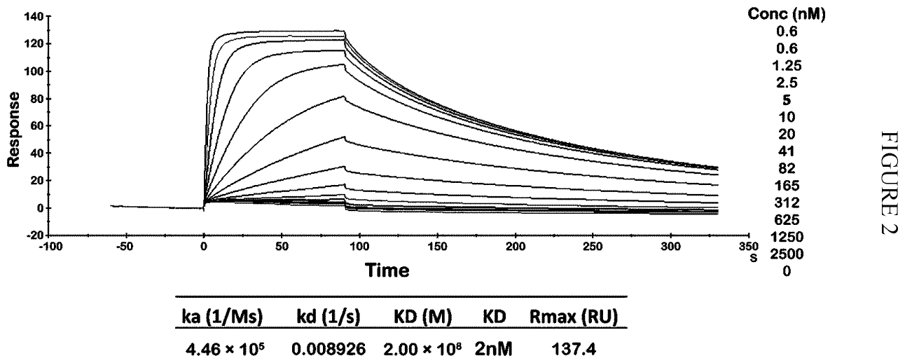

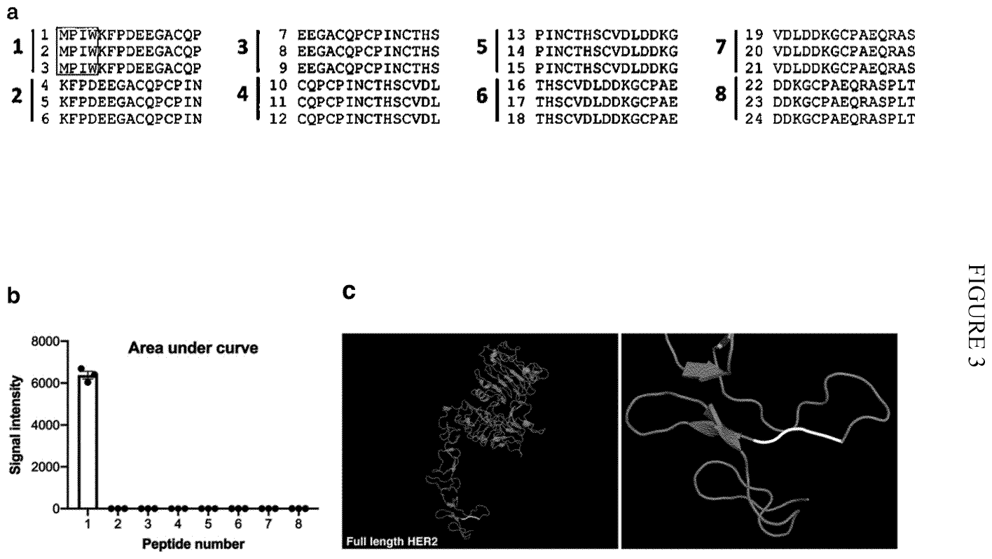

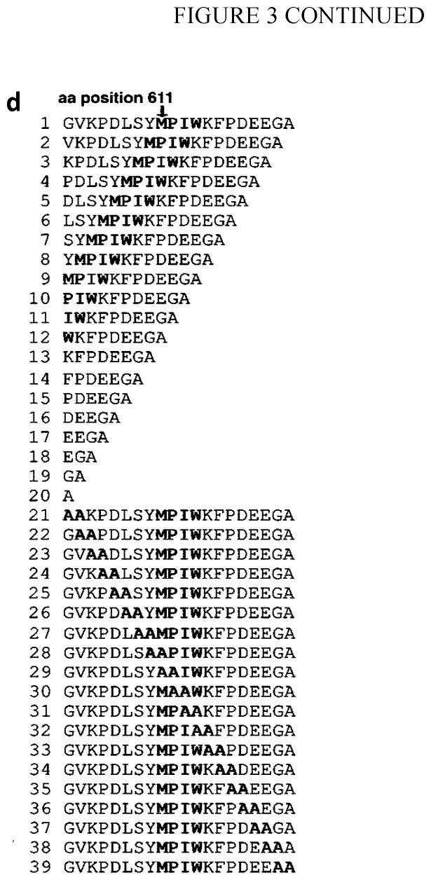

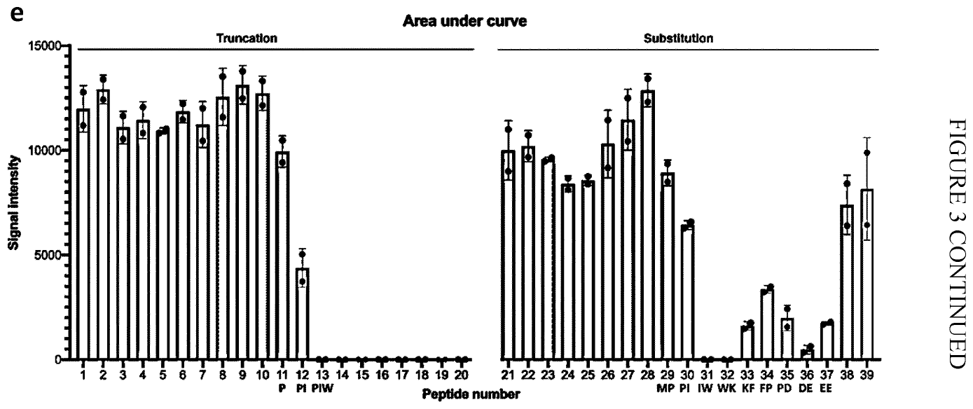

A monoclonal antibody against p95HER2 was thus generated from a rat using standard hybridoma technology. The reactivity and specificity of the antibody was evaluated by flow cytometry based on its binding to a panel of HER2+/− cell lines originating from different solid tumors or haematological malignancies (FIG. 1). Across the different breast cancer cell lines, the antibody demonstrated a strong reactivity to p95HER2-T47D cell line, but it did not bind to the HER2+SK-BR-3 and MDA-MB-468 lines, or to the HER2-T47D, MCF7 and MDA-MB-231 cell lines (FIG. 1). Moreover, the antibody did not bind to the HER2+ lung cancer cell line A549, or to HER2-prostate cancer, pancreatic cancer, lymphoma, and leukemia cell lines (FIG. 1). This showed that the antibody was specific to p95HER2.

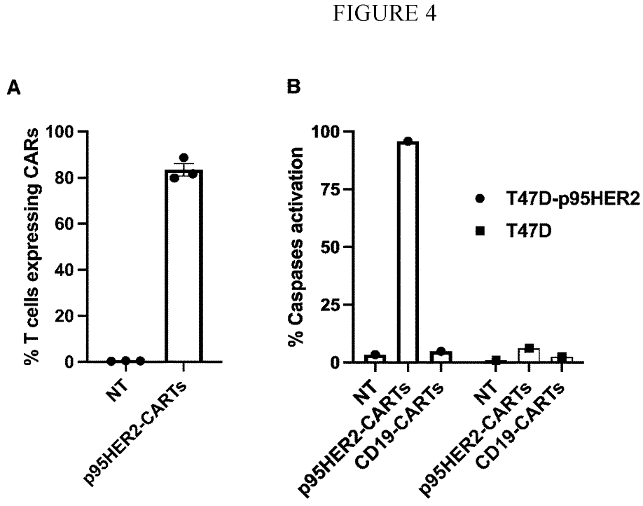

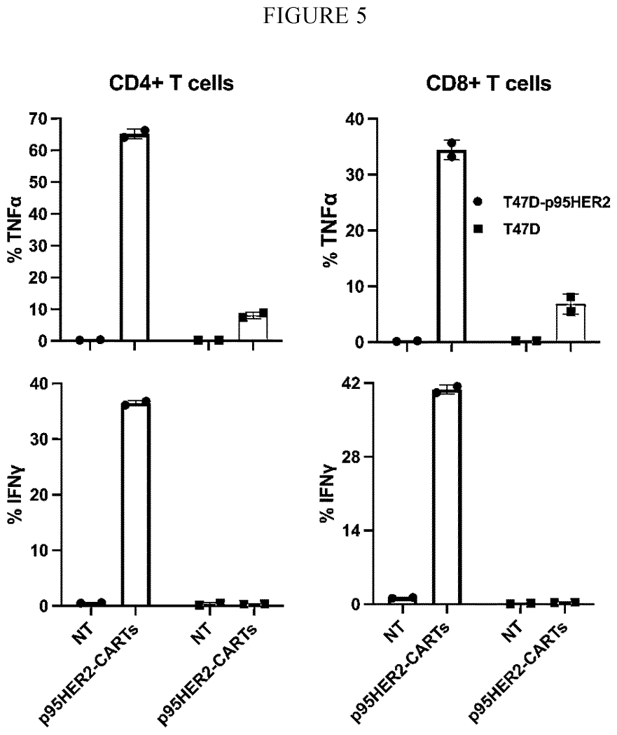

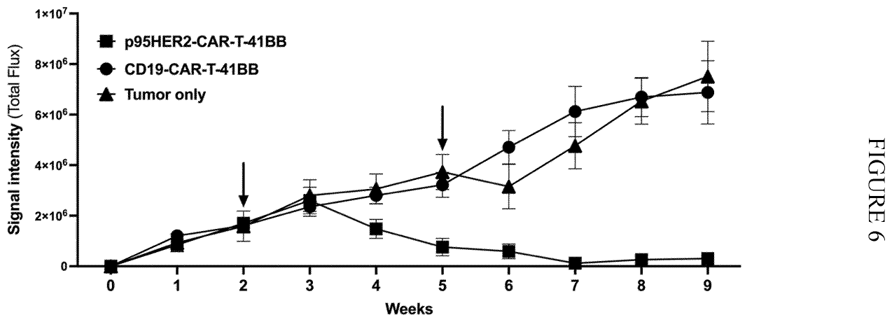

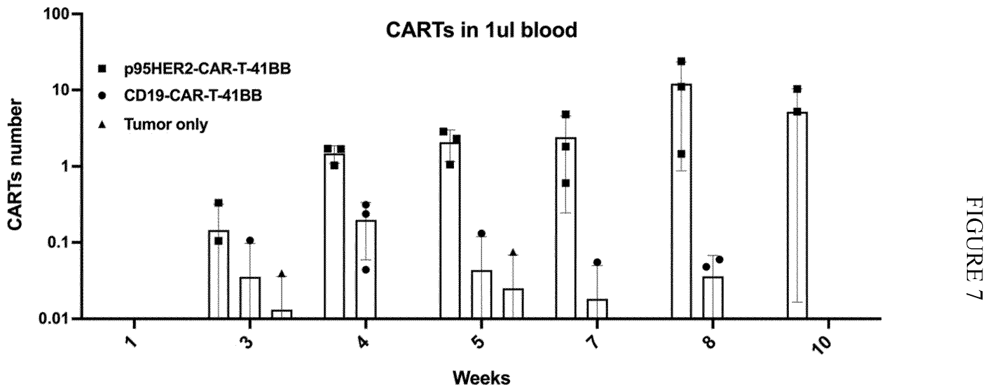

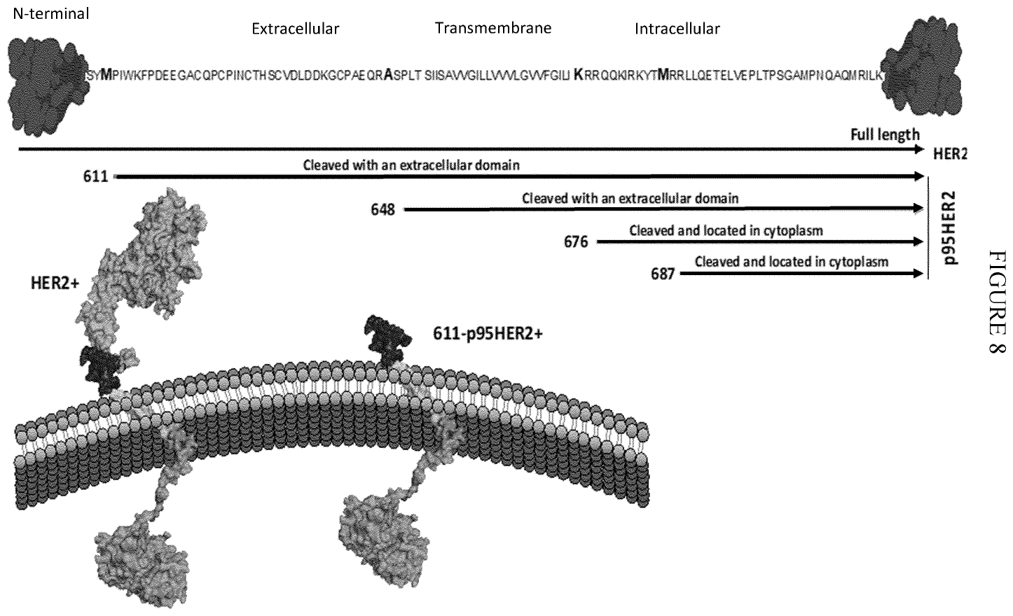

SPR analysis was performed to determine the binding affinity of the antibody to the p95HER2 peptide of SEQ ID NO: 19 (comprising a C-terminal His-tag). The kinetics of the molecular interaction were tested by immobilizing the antibody on a chip and using the extracellular domain of p95HER2 at serial concentrations as the analyte. The control anti-HER2 mAb (ab214275, Abcam, UK) which binds the cytoplasmic domain of HER2 was used as a reference (FIG. 2). The affinity data for the antibody of interest was corrected by subtracting to the control. As expected, the association (ka) rate increased with increasing p95HER2 peptide concentration. The antibody of interest demonstrated a low equilibrium dissociation constant (KD=2 nM) with p95HER2 peptide, representing a high affinity interaction with the maximal binding response (Rmax) at 137 RU (FIG. 2).

### Example 2: Epitope Mapping

To identify the specific epitope recognized by the antibody of interest, we performed epitope mapping using synthetic overlapping peptides that cover the full p95HER2 extracellular domain. In the overlapping peptide strategy, sequential consecutive 15mer peptides overlapping by 4 amino acids were generated (FIG. 3A) and immobilized on cellulose membranes. The results showed that the antibody only bound to peptide 1, which suggested that the 4 amino acid sequence MPIW (SEQ ID NO: 33) was essential for binding (FIGS. 3A & B). This places the binding epitope at positions 611-614 of full-length HER2 (FIG. 3C). The SEQ ID NOs: corresponding to the 15mer peptides shown in FIG. 3A are set out in the following table:

TABLE 3

| SEQ ID NO | Peptide number | Amino acid sequence |
|---|---|---|
| SEQ ID NO: 25 | Peptide 1 | MPIWKFPDEEGACQP |
| SEQ ID NO: 26 | Peptide 2 | KFPDEEGACQPCPIN |
| SEQ ID NO: 27 | Peptide 3 | EEGACQPCPINCTHS |
| SEQ ID NO: 28 | Peptide 4 | CQPCPINCTHSCVDL |
| SEQ ID NO: 29 | Peptide 5 | INCTHSCVDLDDKG |
| SEQ ID NO: 30 | Peptide 6 | THSCVDLDDKGCPAE |
| SEQ ID NO: 31 | Peptide 7 | VDLDDKGCPAEQRAS |
| SEQ ID NO: 32 | Peptide 8 | DDKGCPAEQRASPLT |

We next wanted to investigate if there were any additional amino acids involved in antibody binding and to determine whether the binding epitope was continuous or discontinuous. To this end, we selected the region from glycine-603 to alanine-622 in HER2 that spans the N terminus of the p95HER2 extracellular domain and which contains the MPIW 4mer epitope (SEQ ID NO: 33). We generated peptides that contained either N-terminal truncations for this sequence or sequential peptides that substituted two alanine residues at each position. (FIG. 3D). The SEQ ID NOs: corresponding to the peptides shown in FIG. 3D are set out in the following table:

TABLE 4

| SEQ ID NO | Peptide number | Amino acid sequence |
|---|---|---|
| SEQ ID NO: 35 | Peptide 1 | GVKPDLSYMPIWKFPDEEGA |
| SEQ ID NO: 36 | Peptide 2 | VKPDLSYMPIWKFPDEEGA |
| SEQ ID NO: 37 | Peptide 3 | KPDLSYMPIWKFPDEEGA |
| SEQ ID NO: 38 | Peptide 4 | PDLSYMPIWKFPDEEGA |
| SEQ ID NO: 39 | Peptide 5 | DLSYMPIWKFPDEEGA |
| SEQ ID NO: 40 | Peptide 6 | LSYMPIWKFPDEEGA |
| SEQ ID NO: 41 | Peptide 7 | SYMPIWKFPDEEGA |
| SEQ ID NO: 42 | Peptide 8 | YMPIWKFPDEEGA |
| SEQ ID NO: 43 | Peptide 9 | MPIWKFPDEEGA |
| SEQ ID NO: 44 | Peptide 10 | PIWKFPDEEGA |
| SEQ ID NO: 45 | Peptide 11 | IWKFPDEEGA |
| SEQ ID NO: 46 | Peptide 12 | WKFPDEEGA |
| SEQ ID NO: 47 | Peptide 13 | KFPDEEGA |
| SEQ ID NO: 48 | Peptide 14 | FPDEEGA |
| SEQ ID NO: 49 | Peptide 15 | PDEEGA |
| SEQ ID NO: 50 | Peptide 16 | DEEGA |
| SEQ ID NO: 51 | Peptide 17 | EEGA |
| SEQ ID NO: 52 | Peptide 21 | AAKPDLSYMPIWKFPDEEGA |
| SEQ ID NO: 53 | Peptide 22 | GAAPDLSYMPIWKFPDEEGA |
| SEQ ID NO: 54 | Peptide 23 | GVAADLSYMPIWKFPDEEGA |
| SEQ ID NO: 55 | Peptide 24 | GVKAALSYMPIWKFPDEEGA |
| SEQ ID NO: 56 | Peptide 25 | GVKPAASYMPIWKFPDEEGA |
| SEQ ID NO: 57 | Peptide 26 | GVKPDAAYMPIWKFPDEEGA |
| SEQ ID NO: 58 | Peptide 27 | GVKPDLAAMPIWKFPDEEGA |
| SEQ ID NO: 59 | Peptide 28 | GVKPDLSAAPIWKFPDEEGA |
| SEQ ID NO: 60 | Peptide 29 | GVKPDLSYAAIWKFPDEEGA |
| SEQ ID NO: 61 | Peptide 30 | GVKPDLSYMAAWKFPDEEGA |
| SEQ ID NO: 62 | Peptide 31 | GVKPDLSYMPAAKFPDEEGA |
| SEQ ID NO: 63 | Peptide 32 | GVKPDLSYMPIAAFPDEEGA |
| SEQ ID NO: 64 | Peptide 33 | GVKPDLSYMPIWAAPDEEGA |
| SEQ ID NO: 65 | Peptide 34 | GVKPDLSYMPIWKAADEEGA |
| SEQ ID NO: 66 | Peptide 35 | GVKPDLSYMPIWKFAAEEGA |
| SEQ ID NO: 67 | Peptide 36 | GVKPDLSYMPIWKFPAAEGA |
| SEQ ID NO: 68 | Peptide 37 | GVKPDLSYMPIWKFPDAAGA |
| SEQ ID NO: 69 | Peptide 38 | GVKPDLSYMPIWKFPDEAAA |
| SEQ ID NO: 70 | Peptide 39 | GVKPDLSYMPIWKFPDEEAA |

N-terminal truncations showed that methionine-611 does not playing a key role in antibody binding, as the first reduction of binding was observed only after the loss of proline-612. Binding was further decreased with the deletion of isoleucine-613, and binding was lost completely when Tryptophan-614 was removed (peptide 11) (FIG. 3E). We observed the same results with the double-alanine substitutions where antibody binding was lost with the substitution of the MPIW (SEQ ID NO: 33) epitope. However, these studies also revealed that substituting the amino acids immediately adjacent to the original epitope, from positions lysine-622 to glutamate-621, also play a role in antibody binding (FIG. 3E). Therefore, from these studies, we conclude that the binding epitope of the antibody is continuous and has the sequence PIWKFPDEE (SEQ ID NO: 17). The 3D structure of full-length HER2 and p95HER2 show that this epitope is hidden in full-length HER2.

### Example 3: Expression Level and In Vitro Activity of the Invention

To generate p95HER2-CAR-Ts from the antibody, activated T cells were transduced with a CAR construct comprising RQR8, signal peptide, p95HER2 scFv (derived from the antibody), CD8α hinge, CD8α transmembrane domain, 4-1BB costimulatory domain and CD3ζ signaling domain within a retrovirus expression vector. RQR8 is a compact epitope-based marker/suicide gene, containing minimal target epitopes from CD34 and CD20 antigens. This gene is under the same promotor as the CAR and it is separated from the CAR by a self-cleaving protein named 2A. By detecting RQR8 (using the anti-CD34 antibody-QBEND) we evaluated the CAR expression (FIG. 4A). To investigate p95HER2-CAR-T functionality in vitro, T cells transduced with p95HER2-CAR construct and non-transduced T cells were co-cultured with relevant (p95HER2+) and irrelevant (p95HER−) target cells. The cytotoxicity and cytokine production capacity of p95HER2-CAR-Ts were evaluated through different assays. It was observed that p95HER2-CAR-Ts could induce apoptosis in p95HER2+ target cells (FIG. 4B) and secrete inflammatory cytokines TNFα and IFNγ (FIG. 5).

### Example 4: How to Treat a Model Cancer in Mice Based on the Invention

The p95HER2-CAR-Ts that were tested successfully in vitro (Example 3) were examined further in vivo. A p95HER2 positive orthopedic xenograft mouse model has been established in the group by the orthopedic implementation of p95HER2-T47D cells into the mammary fat pad. CAR-Ts were injected 2 times intravenously (2 and 5 weeks after tumor implantation), 5 million CAR-Ts at each time point. Tumor growth was evaluated by bioluminescence in vivo imaging during the time-course of the treatment. Notable tumor reduction was observed as early as 2 weeks after the first p95HER2-CAR-T injection and maximum tumor control (tumor elimination) was observed 5 weeks after the first p95HER2-CAR-T injection (FIG. 6). p95HER2-CAR-Ts could persist and expand in vivo for a relatively long period (10 weeks) after the first injection (FIG. 7).

In the Figures and examples, both “p95HER2-CAR-Ts” and “p95HER-CAR-T-41BB” means T-cells expressing the CAR of SEQ ID NO: 15.

### Example 5: Antibody Humanization

The IMGT VH and VL CDRs (SEQ ID NO: 80-84) of the invention as set out in Table 2 were grafted into human germline sequences using a CDR grafting algorithm. Table 5 below sets out the percentage identity of the parental and humanized sequences to the selected human germline sequences.

TABLE 5

| Name | % identity to human | Human germline |
|---|---|---|
| Parental VH | 70.4% | — |
| hu-VH1 (SEQ ID NO: 72) | 79.6% | IGHV3-72*01 |
| hu-VH2 (SEQ ID NO: 73) | 81.6% | IGHV3-72*01 |
| hu-VH3 (SEQ ID NO: 74) | 69.4% | IGHV4-59*01 |
| hu-VH4 (SEQ ID NO: 75) | 74.0% | IGHV4-59*01 |
| Parental VL | 80.2% | — |
| hu-VL1 (SEQ ID NO: 76) | 89.1% | IGKV4-1*01 |
| hu-VL2 (SEQ ID NO: 77) | 90.1% | IGKV4-1*01 |
| hu-VL3 (SEQ ID NO: 78) | 84.2% | IGKV2-28*01 |
| hu-VL4 (SEQ ID NO: 79) | 85.1% | IGKV2-28*01 |

### Sequence Liability Analysis

To ensure that no highly undesirable sequence liabilities had been introduced into the humanized sequences the parental and humanized sequences were run through an Absolute Antibody sequence liability tool. Sequence liabilities of most concern are glycosylation sites and free cysteines, none of which are present in these sequences. Motifs for deamidation and isomerization are present in the sequences. These modifications are designated as high risk, and can cause disruption during manufacturing, but they are often manageable. If desired, it may be possible to remove these motifs through mutagenesis. Medium and low risk sequence liabilities are also present but are rarely reported to cause problems in an antibody manufacturing process.

### Antibody Cloning

A total of 4 humanized heavy chains and 4 humanized light chains were designed (SEQ ID NOs: 72-79). Each of these were synthesised separately and cloned into human IgG1 heavy chain and human kappa light chain expression vectors respectively. At the point of transfection all possible combinations of the humanized sequences were made to create a total of 16 different humanized antibodies, which are set out in Table 1 above.

### Antibody Expression and Purification

Antibodies were expressed and purified by Protein A. Purified protein was buffer exchanged and concentrated. All antibodies were expressed and all the purified products looked as expected under non-reducing and reducing SDS-PAGE.

### Aggregation Analysis

Purified antibodies were analysed for aggregation and fragmentation by SEC-HPLC. All purified antibodies showed good monomer content.

1. A binding molecule which specifically binds p95HER2 comprising the amino acid sequence set forth in SEQ ID NO: 17,
comprising a light chain variable domain (VL) and a heavy chain variable domain (VH) which together form an antigen binding unit,
wherein the VL comprises three complementarity determining regions (CDRs): CDR1, CDR2 and CDR3, which respectively comprise the amino acid sequences SEQ ID NOs: 1, 2 and 3; and
wherein the VH comprises three CDRs; CDR1, CDR2 and CDR3, which respectively comprise the amino acid sequences SEQ ID NOs: 4, 5 and 6.

2. The binding molecule according to claim 1,
wherein the VH comprises the amino acid sequence set forth in SEQ ID NO: 7, or a sequence with at least 90% identity thereto, and
wherein the VL comprises the amino acid sequence set forth in SEQ ID NO: 8, or a sequence with at least 90% identity thereto.

3. The binding molecule according to claim 1 or 2, wherein the molecule is an antibody or fragment thereof.

4. The binding molecule according to claim 1 or 2, wherein the antigen binding unit is a scFv.

5. A Chimeric Antigen Receptor (CAR) comprising an antigen binding unit as defined in any one of claim 1, 2 or 4.

6. The CAR according to claim 5, comprising a human CD8α hinge of SEQ ID NO: 11 or a sequence with at least 90% identity thereto.

7. The CAR according to claim 6, comprising from N-terminal to C-terminal, a human CD8α hinge, a human CD8α transmembrane domain, a human 4-1BB costimulatory domain and a human CD3ζ signaling domain.

8. A nucleic acid encoding the binding molecule according to any one of claims 1 to 4 or the CAR according to any one of claims 5 to 7.

9. A vector comprising the nucleic acid of claim 8.

10. A cytotoxic immune cell expressing a CAR according to any one of claims 5 to 7 in its cell membrane.

11. The cytotoxic immune cell according to claim 10, wherein the cell is a cytotoxic T cell or an NK cell.

12. A pharmaceutical composition comprising a binding molecule according to any one of claims 1 to 4.

13. A pharmaceutical composition comprising a nucleic acid according to claim 8 or a vector according to claim 9.

14. A pharmaceutical composition comprising a cytotoxic immune cell according to claim 10 or 11.

15. A method of treatment of cancer in a human patient comprising the step of administering a binding molecule according to any one of claims 1 to 4, a cytotoxic immune cell according to claim 10 or 11 or a pharmaceutical composition according to any one of claims 12 to 14.

16. A method of treatment of cancer in a human patient comprising the steps:
a. obtaining a sample comprising cancer cells from the patient;
b. analysing whether the cancer cells express p95HER2 by contacting the cells ex vivo with a binding molecule as defined in any one of claims 1 to 4 further comprising a moiety suitable for detection, and
c. administering a chemotherapy to the patient if the cancer cells are p95HER2 positive.

17. A method of diagnosing cancer comprising the steps
a. obtaining a sample comprising cells from a human patient;
b. analysing whether the cells express p95HER2 by contacting the cells ex vivo with a binding molecule as defined in any one of claims 1 to 4, wherein the protein comprises a moiety suitable for detection; and
c. diagnosing the patient with cancer if the cells express p95HER2.

18. A binding molecule according to any one of claims 1 to 4, a CAR according to any one of claims 5 to 7, a cytotoxic immune cell according to claim 10 or 11 or a pharmaceutical composition according to any one of claims 12 to 14 for use in therapy.

19. A binding molecule according to any one of claims 1 to 4, a CAR according to any one of claims 5 to 7, a cytotoxic immune cell according to claim 10 or 11 or a pharmaceutical composition according to any one of claims 12 to 14 for use in the treatment of cancer, wherein the cancer expresses p95HER2 comprising the amino acid sequence set forth in SEQ ID NO: 17.

20. The binding molecule, CAR, cytotoxic immune cell or pharmaceutical composition for use according to claim 19, wherein the cancer is breast cancer.

21. A method of diagnosing cancer in a subject, the method comprising:
(a) contacting a sample of cells from the subject with a binding molecule as defined in any one of claims 1 to 4, wherein the binding molecule further comprises a detection moiety;
(b) determining whether the cells express p95HER2; and
(c) if the cells express p95HER2, diagnosing the patient with cancer.

22. A binding molecule which specifically binds p95HER2 comprising the amino acid sequence set forth in SEQ ID NO: 17,
comprising a humanized light chain variable domain (VL) and a humanized heavy chain variable domain (VH) which together form an antigen binding unit,
wherein the VL comprises an amino acid sequence as set forth in any one of SEQ ID NOs: 72 to 75; and
wherein the VH comprises an amino acid sequence as set forth in any one of SEQ ID NOs: 76 to 79.


SEQUENCE LISTING (90 sequences)

SEQ ID NO: 1 - protein, 17 aa, Rattus sp.
  source 1..17: note = Murine, Kabat VL CDR1
KSSQSLLSSGNQKNNLA

SEQ ID NO: 2 - protein, 7 aa, Rattus sp.
  source 1..7: note = Murine, Kabat VL CDR2
YASTRQS

SEQ ID NO: 3 - protein, 9 aa, Rattus sp.
  source 1..9: note = Murine, Kabat VL CDR3
LQHYSSPYT

SEQ ID NO: 4 - protein, 5 aa, Rattus sp.
  source 1..5: note = Murine, Kabat VH CDR1
DYFMN

SEQ ID NO: 5 - protein, 19 aa, Rattus sp.
  source 1..19: note = Murine, Kabat VH CDR2
QIRNKNYNYATYFAESLEG

SEQ ID NO: 6 - protein, 5 aa, Rattus sp.
  source 1..5: note = Murine, Kabat VH CDR3
LRYDY

SEQ ID NO: 7 - protein, 116 aa, Rattus sp.
  source 1..116: note = Murine
EVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSS

SEQ ID NO: 8 - protein, 113 aa, Rattus sp.
  source 1..113: note = Murine
DIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELK

SEQ ID NO: 9 - protein, 249 aa, synthetic construct
  source 1..249: note = scFv, VH-linker-VL
EVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSSGGGGSGGGGSAGGGSGGGGSDIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELK

SEQ ID NO: 10 - protein, 249 aa, synthetic construct
  source 1..249: note = scFv, VL-linker-VH
DIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELKGGGGSGGGGSAGGGSGGGGSEVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSS

SEQ ID NO: 11 - protein, 45 aa, Homo sapiens
SDPTTTPAPRPPTPAPTIASQPLSLRPEACRPAAGGAVHTRGLDF

SEQ ID NO: 12 - protein, 27 aa, Homo sapiens
ACDIYIWAPLAGTCGVLLLSLVITLYC

SEQ ID NO: 13 - protein, 42 aa, Homo sapiens
KRGRKKLLYIFKQPFMRPVQTTQEEDGCSCRFPEEEEGGCEL

SEQ ID NO: 14 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 475 aa, synthetic construct
  source 1..475: note = Full length second generation p95HER2-CAR
EVQILETGGGLVKPGGSLRLSCATSGFNFNDYFMNWVRQAPGKGLEWIAQIRNKNYNYATYFAESLEGRFTISRDDSKSSVYLQVNSLRAEDTALYYCTELRYDYWGQGVMVTVSSGGGGSGGGGSAGGGSGGGGSDIVMTQSPFSLAVSEGEMVTINCKSSQSLLSSGNQKNNLAWYQQKPGQSPKLLIYYASTRQSGVPDRFIGSGSGTDFTLTISDVQAEDLADYYCLQHYSSPYTFGAGTKLELKSDPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 20 aa, synthetic construct
  source 1..20: note = Linker peptide
MESQTQALISLLLWVYGTYG

SEQ ID NO: 17 - protein, 9 aa, Homo sapiens
  source 1..9: note = Target epitope
PIWKFPDEE

SEQ ID NO: 18 - protein, 20 aa, synthetic construct
  source 1..20: note = Linker
GGGGSGGGGSAGGGSGGGGS

SEQ ID NO: 19 - protein, 48 aa, synthetic construct
  source 1..48: note = SPR Epitope
MPIWKFPDEEGACQPCPINCTHSCVDLDDKGCPAEQRASPLTHHHHHH

SEQ ID NO: 20 - protein, 645 aa, Homo sapiens
MPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 21 - protein, 5 aa, synthetic construct
  source 1..5: note = Linker
GGGGS

SEQ ID NO: 22 - protein, 15 aa, synthetic construct
  source 1..15: note = Linker
GGGGSGGGGSGGGGS

SEQ ID NO: 23 - protein, 20 aa, synthetic construct
  source 1..20: note = Linker
GGGGSGGGGSGGGGSGGGGS

SEQ ID NO: 24 - protein, 25 aa, synthetic construct
  source 1..25: note = Linker
GGGGSGGGGSGGGGSGGGGSGGGGS

SEQ ID NO: 25 - protein, 15 aa, synthetic construct
  source 1..15: note = Peptide
MPIWKFPDEEGACQP

SEQ ID NO: 26 - protein, 15 aa, synthetic construct
  source 1..15: note = Peptide
KFPDEEGACQPCPIN

SEQ ID NO: 27 - protein, 15 aa, synthetic construct
  source 1..15: note = Peptide
EEGACQPCPINCTHS

SEQ ID NO: 28 - protein, 15 aa, synthetic construct
  source 1..15: note = Peptide
CQPCPINCTHSCVDL

SEQ ID NO: 29 - protein, 14 aa, synthetic construct
  source 1..14: note = Peptide
INCTHSCVDLDDKG

SEQ ID NO: 30 - protein, 15 aa, synthetic construct
  source 1..15: note = Peptide
THSCVDLDDKGCPAE

SEQ ID NO: 31 - protein, 15 aa, synthetic construct
  source 1..15: note = Peptide
VDLDDKGCPAEQRAS

SEQ ID NO: 32 - protein, 15 aa, synthetic construct
  source 1..15: note = Peptide
DDKGCPAEQRASPLT

SEQ ID NO: 33 - protein, 4 aa, synthetic construct
  source 1..4: note = Binding epitope
MPIW

SEQ ID NO: 34 - protein, 6 aa, synthetic construct
  source 1..6: note = Binding epitope
KFPDEE

SEQ ID NO: 35 - protein, 20 aa, synthetic construct
  source 1..20: note = Peptide
GVKPDLSYMPIWKFPDEEGA

SEQ ID NO: 36 - protein, 19 aa, synthetic construct
  source 1..19: note = Peptide
VKPDLSYMPIWKFPDEEGA

SEQ ID NO: 37 - protein, 18 aa, synthetic construct
  source 1..18: note = Peptide
KPDLSYMPIWKFPDEEGA

SEQ ID NO: 38 - protein, 17 aa, synthetic construct
  source 1..17: note = Peptide
PDLSYMPIWKFPDEEGA

SEQ ID NO: 39 - protein, 16 aa, synthetic construct
  source 1..16: note = Peptide
DLSYMPIWKFPDEEGA

SEQ ID NO: 40 - protein, 15 aa, synthetic construct
  source 1..15: note = Peptide
LSYMPIWKFPDEEGA

SEQ ID NO: 41 - protein, 14 aa, synthetic construct
  source 1..14: note = Peptide
SYMPIWKFPDEEGA

SEQ ID NO: 42 - protein, 13 aa, synthetic construct
  source 1..13: note = Peptide
YMPIWKFPDEEGA

SEQ ID NO: 43 - protein, 12 aa, synthetic construct
  source 1..12: note = Peptide
MPIWKFPDEEGA

SEQ ID NO: 44 - protein, 11 aa, synthetic construct
  source 1..11: note = Peptide
PIWKFPDEEGA

SEQ ID NO: 45 - protein, 10 aa, synthetic construct
  source 1..10: note = Peptide
IWKFPDEEGA

SEQ ID NO: 46 - protein, 9 aa, synthetic construct
  source 1..9: note = Peptide
WKFPDEEGA

SEQ ID NO: 47 - protein, 8 aa, synthetic construct
  source 1..8: note = Peptide
KFPDEEGA

SEQ ID NO: 48 - protein, 7 aa, synthetic construct
  source 1..7: note = Peptide
FPDEEGA

SEQ ID NO: 49 - protein, 6 aa, synthetic construct
  source 1..6: note = Peptide
PDEEGA

SEQ ID NO: 50 - protein, 5 aa, synthetic construct
  source 1..5: note = Peptide
DEEGA

SEQ ID NO: 51 - protein, 4 aa, synthetic construct
  source 1..4: note = Peptide
EEGA

SEQ ID NO: 52 - protein, 20 aa, synthetic construct
  source 1..20: note = Peptide
AAKPDLSYMPIWKFPDEEGA

SEQ ID NO: 53 - protein, 20 aa, synthetic construct
  source 1..20: note = Peptide
GAAPDLSYMPIWKFPDEEGA

SEQ ID NO: 54 - protein, 20 aa, synthetic construct
  source 1..20: note = Peptide
GVAADLSYMPIWKFPDEEGA

SEQ ID NO: 55 - protein, 20 aa, synthetic construct
  source 1..20: note = Peptide
GVKAALSYMPIWKFPDEEGA

SEQ ID NO: 56 - protein, 20 aa, synthetic construct
  source 1..20: note = Peptide
GVKPAASYMPIWKFPDEEGA

SEQ ID NO: 57 - protein, 20 aa, synthetic construct
  source 1..20: note = Peptide
GVKPDAAYMPIWKFPDEEGA

SEQ ID NO: 58 - protein, 20 aa, synthetic construct
  source 1..20: note = Peptide
GVKPDLAAMPIWKFPDEEGA

SEQ ID NO: 59 - protein, 20 aa, synthetic construct
  source 1..20: note = Peptide
GVKPDLSAAPIWKFPDEEGA

SEQ ID NO: 60 - protein, 20 aa, synthetic construct
  source 1..20: note = Peptide
GVKPDLSYAAIWKFPDEEGA

SEQ ID NO: 61 - protein, 20 aa, synthetic construct
  source 1..20: note = Peptide
GVKPDLSYMAAWKFPDEEGA

SEQ ID NO: 62 - protein, 20 aa, synthetic construct
  source 1..20: note = Peptide
GVKPDLSYMPAAKFPDEEGA

SEQ ID NO: 63 - protein, 20 aa, synthetic construct
  source 1..20: note = Peptide
GVKPDLSYMPIAAFPDEEGA

SEQ ID NO: 64 - protein, 20 aa, synthetic construct
  source 1..20: note = Peptide
GVKPDLSYMPIWAAPDEEGA

SEQ ID NO: 65 - protein, 20 aa, synthetic construct
  source 1..20: note = Peptide
GVKPDLSYMPIWKAADEEGA

SEQ ID NO: 66 - protein, 20 aa, synthetic construct
  source 1..20: note = Peptide
GVKPDLSYMPIWKFAAEEGA

SEQ ID NO: 67 - protein, 20 aa, synthetic construct
  source 1..20: note = Peptide
GVKPDLSYMPIWKFPAAEGA

SEQ ID NO: 68 - protein, 20 aa, synthetic construct
  source 1..20: note = Peptide
GVKPDLSYMPIWKFPDAAGA

SEQ ID NO: 69 - protein, 20 aa, synthetic construct
  source 1..20: note = Peptide
GVKPDLSYMPIWKFPDEAAA

SEQ ID NO: 70 - protein, 20 aa, synthetic construct
  source 1..20: note = Peptide
GVKPDLSYMPIWKFPDEEAA

SEQ ID NO: 71 - protein, 108 aa, Homo sapiens
SYMPIWKFPDEEGACQPCPINCTHSCVDLDDKGCPAEQRASPLTSIISAVVGILLVVVLGVVFGILIKRRQQKIRKYTMRRLLQETELVEPLTPSGAMPNQAQMRILK

SEQ ID NO: 72 - protein, 116 aa, synthetic construct
  source 1..116: note = Hu-VH1
EVQIVESGGGLVQPGGSLRLSCATSGFNFNDYFMNWVRQAPGKGLEWIAQIRNKNYNYATYFAESVKGRFTISRDDSKSSVYLQMNSLKTEDTAVYYCTELRYDYWGQGTMVTVSS

SEQ ID NO: 73 - protein, 116 aa, synthetic construct
  source 1..116: note = Hu-VH2
EVQIVESGGGLVQPGGSLRLSCATSGFNFNDYFMNWVRQAPGKGLEWVAQIRNKNYNYATYFAESVKGRFTISRDDSKNSVYLQMNSLKTEDTAVYYCTELRYDYWGQGTMVTVSS

SEQ ID NO: 74 - protein, 116 aa, synthetic construct
  source 1..116: note = Hu-VH3
QVQIQESGPGLVKPSETLSLTCTTSGFNFNDYFMNWVRQPPGKGLEWIAQIRNKNYNYATYFAESLKSRFTISRDDSKSSVSLKLSSVTAADTAVYYCTELRYDYWGQGTMVTVSS

SEQ ID NO: 75 - protein, 116 aa, synthetic construct
  source 1..116: note = Hu-VH4
QVQIQESGPGLVKPSETLSLTCTTSGFNFNDYFMNWIRQPPGKGLEWIAQIRNKNYNYATYFAESLKSRVTISRDDSKNQVSLKLSSVTAADTAVYYCTELRYDYWGQGTMVTVSS

SEQ ID NO: 76 - protein, 113 aa, synthetic construct
  source 1..113: note = Hu-VL1
DIVMTQSPDSLAVSLGERATINCKSSQSLLSSGNQKNNLAWYQQKPGQPPKLLIYYASTRQSGVPDRFSGSGSGTDFTLTISSLQAEDVADYYCLQHYSSPYTFGGGTKLEIK

SEQ ID NO: 77 - protein, 113 aa, synthetic construct
  source 1..113: note = Hu-VL2
DIVMTQSPDSLAVSLGERATINCKSSQSLLSSGNQKNNLAWYQQKPGQPPKLLIYYASTRQSGVPDRFSGSGSGTDFTLTISSLQAEDVAVYYCLQHYSSPYTFGGGTKLEIK

SEQ ID NO: 78 - protein, 113 aa, synthetic construct
  source 1..113: note = Hu-VL3
DIVMTQSPLSLPVTPGEPASISCRSSQSLLSSGNQKNNLAWYLQKPGQSPQLLIYYASTRQSGVPDRFSGSGSGTDFTLKISRVEAEDVGDYYCLQHYSSPYTFGGGTKLEIK

SEQ ID NO: 79 - protein, 113 aa, synthetic construct
  source 1..113: note = Hu-VL4
DIVMTQSPLSLPVTPGEPASISCRSSQSLLSSGNQKNNLAWYLQKPGQSPQLLIYYASTRQSGVPDRFSGSGSGTDFTLKISRVEAEDVGVYYCLQHYSSPYTFGGGTKLEIK

SEQ ID NO: 80 - protein, 8 aa, Rattus sp.
  source 1..8: note = Murine, IMGT VH CDR1
GFNFNDYF

SEQ ID NO: 81 - protein, 10 aa, Rattus sp.
  source 1..10: note = Murine, IMGT VH CDR2
IRNKNYNYAT

SEQ ID NO: 82 - protein, 7 aa, Rattus sp.
  source 1..7: note = Murine, IMGT VH CDR3
TELRYDY

SEQ ID NO: 83 - protein, 12 aa, Rattus sp.
  source 1..12: note = Murine, IMGT VL CDR1
QSLLSSGNQKNN

SEQ ID NO: 84 - protein, 9 aa, Rattus sp.
  source 1..9: note = Murine, IMGT VL CDR3
LQHYSSPYT

SEQ ID NO: 85 - protein, 7 aa, Rattus sp.
  source 1..7: note = Murine, Chothia VH CDR1
GFNFNDY

SEQ ID NO: 86 - protein, 8 aa, Rattus sp.
  source 1..8: note = Murine, Chothia VH CDR2
RNKNYNYA

SEQ ID NO: 87 - protein, 5 aa, Rattus sp.
  source 1..5: note = Murine, Chothia VH CDR3
LRYDY

SEQ ID NO: 88 - protein, 17 aa, Rattus sp.
  source 1..17: note = Murine, Chothia VL CDR1
KSSQSLLSSGNQKNNLA

SEQ ID NO: 89 - protein, 7 aa, Rattus sp.
  source 1..7: note = Murine, Chothia VL CDR2
YASTRQS

SEQ ID NO: 90 - protein, 9 aa, Rattus sp.
  source 1..9: note = Murine, Chothia VL CDR3
LQHYSSPYT